(19) United States
(12) Patent Application Publication
(10) Pub. No.: US 2009/0264802 A1
(43) Pub. Date: Oct. 22, 2009

(54) Cervical collar

(76) Inventor: Jyi Feng Chen, Taipei County (TW)

Correspondence Address: BACON & THOMAS, PLLC, 625 SLATERS LANE, FOURTH FLOOR, ALEXANDRIA, VA, 22314-1176, US

(21) Appl. No.: 12/081,424

(22) Filed: Apr. 16, 2008

(51) Int. Cl. A61F 5/01

(52) U.S. Cl. 602/18

(57) ABSTRACT

A cervical collar is disclosed. The cervical collar includes a main collar structure and at least one flexible adjustment member. The main collar structure can be configured to attach the upper chest and the shoulder of the patient and support the head of the patient. The main collar structure further has at least one concave portion. The flexible adjustment member can be disposed to the concave portions and is provided for securing the main collar structure at the back of the neck of the patient and can be configured to form a treatment region at the back of the neck of the patient, thereby conveniently caring the patient at the treatment region.

### FIELD OF THE INVENTION

The present invention generally relates to a cervical collar, and more particularly to the cervical collar that has a treatment region formed on the back of the neck of the patient to conveniently care the patient after surgical treatment is done.

### BACKGROUND OF THE INVENTION

When a human body suffers injuries, wounds must be treated, and sometimes, those injured people needs to wear different protectors for immobilizing their limbs or torsos before going to hospitals or taking further treatment. For example, a conventional cervical collar is one of protectors to immobilize a patient's neck and head. The conventional cervical collar may also use at least one fastening band to ensure that the conventional cervical collar can efficiently stabilize the patient's head and neck to prevent the patient from suffering pains. However, the patient may feel uncomfortable with the conventional cervical collar since the conventional cervical collar is usually compactness, and closely covers with the patient's neck and stresses the wound to cause hot and windless and/or pain. Moreover, when some surgical operations may be performed on the back of the neck, the foregoing drawback of the conventional cervical collar may result in difficultly care the wound or the treatment region from the neck of the patient.

To overcome the foregoing shortcoming, the inventor of the present invention based on years of experience in the related field to conduct extensive researches and experiments, and finally invented a cervical collar as a method or a basis for resolving the foregoing drawback.

### SUMMARY OF THE INVENTION

Briefly, it is a first object of the present invention to provide a cervical collar for a patient's neck and helping post-surgical treatment. The cervical collar includes a main collar structure and at least one flexible adjustment member. The main collar structure has a concave portion for accommodating the flexible adjustment member. The main collar structure can be configured to attach the upper chest and the shoulder of the patient and support the head of the patient. The flexible adjustment member can be provided for securing the main collar structure at the back of the neck of the patient and can be configured to form a treatment region at the back of the neck of the patient, thereby conveniently caring the patient at the treatment region.

Since the flexible adjustment member is used at the back of the neck of the patient and the treatment region at the back of the neck is formed through the flexible adjustment member, doctors/nurses can conveniently care the patient from the back of the neck after surgical treatment is done. Further, the wound may not be covered or stressed, and can be ventilated to speed up the degree of rehabilitation.

Moreover, the cervical collar has the advantages of light weight, beauty, simple wearing, easy adjustment, better fixity and comfortable. The most important thing is that the cervical collar of the present invention does not influence the post-surgical treatment by comparing with the conventional cervical collar.

A second objective of the present invention is to provide a cervical collar for a patient's neck and encircling the neck between the patient's head and body to immobilize the patient's head and neck. The cervical collar includes a main collar structure and at least one flexible adjustment member. The main collar structure further includes a flexible metal wire, a hollow tube and a fabric. The hollow tube has a hollow space for accommodating the flexible metal wire. The fabric can be adhered to a surface of the hollow tube through an adhesive material to attach the patient. Since the flexible metal wire, the hollow tube and the cloth are assembled, the main collar structure is made. The flexible adjustment member then is used to secure the main collar structure at the back of the neck of the patient to form a treatment region at the back of the neck of the patient.

To make it easier for our examiner to understand the object of the invention, its innovative features and performance, a detailed description and technical characteristics of the present invention are described together with the drawings as follows.

### BRIEF DESCRIPTION OF THE DRAWINGS

FIG. 1A, 1B are schematic diagrams illustrating a cervical collar worn on a patient's neck of the present invention;

FIG. 2A is a side view illustrating a cervical collar according to an embodiment of the present invention;

FIG. 2B is a rear view illustrating a cervical collar according to an embodiment of the present invention;

FIG. 3 is a schematic diagram illustrating the main collar structure of a cervical collar equipped with a U-shaped fastening band of the present invention;

FIG. 4 is a detail schematic diagram illustrating a cervical collar of the present invention; and

FIG. 5 is a flowchart illustrating a method of assembling a cervical collar according to an embodiment of the present invention.

### DETAILED DESCRIPTION OF THE PREFERRED EMBODIMENTS

Referring to the related figures for the cervical collar according to a preferred embodiment of the present invention, wherein the same elements are described by the same reference numerals.

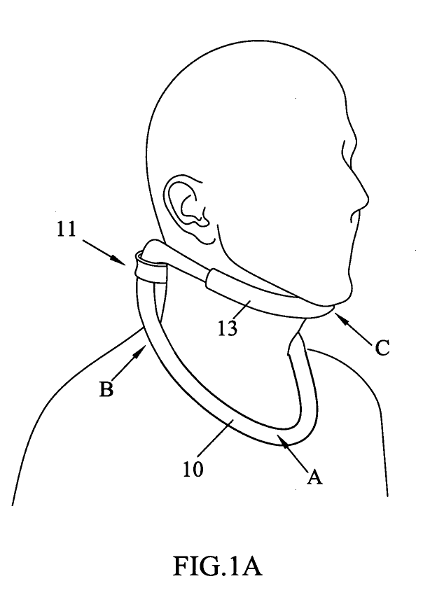

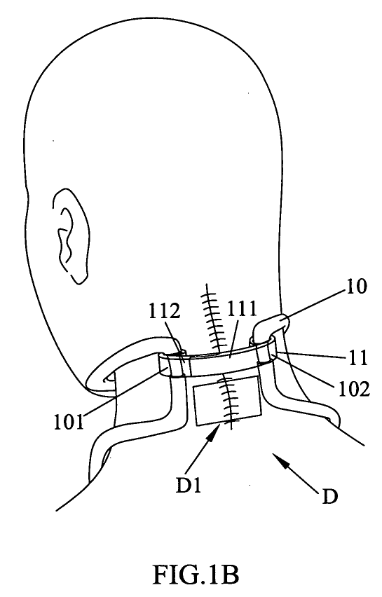

Referring to FIG. 1A, 1B for the schematic diagrams illustrate a cervical collar worn on a patient's neck of the present invention. The cervical collar can help post-surgical treatment, and includes a main collar structure 10 and at least one flexible adjustment member 11. The main collar structure 10 has concave portions 101, 102, and is configured to attach the upper chest A and the shoulder B of the patient and support the head C of the patient as shown in FIG. 1A. The main collar structure 10, which leans against the upper chest A of the patient, is further equipped with a soft mat 13 to make patient more comfortable. The flexible adjustment member 11 is disposed to the concave portions 101, 102 and provided for securing the main collar structure 10 at the back of the neck D of the patient and configured to form a treatment region D1 at the back of the neck D so as to conveniently care the patient at the treatment region D1 (referred to dashed lines) as shown in FIG. 1B.

The main collar structure 10 is preferably composed of a flexible metal wire, a hollow tube and a fabric. The flexible metal wire is preferably steel wire. The flexible metal wire can also be other kinds of metals with better flexibility. The steel wire can be preferably made of cast iron. The diameter of the flexible metal wire can be preferably between 0.5 mm (millimeter) and 7 mm. The hollow tube further has a hollow space that accommodates the flexible metal wire, and is made of rubber or other flexible material. The internal diameter of the hollow tube can be preferably between 4 mm and 10 mm, and the external diameter of the hollow tube can be preferably between 8 mm and 15 mm. The fabric can be preferably nonwoven or other material having waterproof function. The flexible adjustment member 11 is preferably a fastening band, and can be Velcro® or other snap fasteners. The flexible adjustment member 11 can be made of nylon, polymer composition material or other material capable of repeatedly adhering to and tearing off from the main collar structure 10. The flexible adjustment member 11 has a fastening band 111 and a fastened band 112. Both the fastening band 111 and the fastened band 112 are circularly disposed to the concave portions 101, 102. The fastening band 111 can be further provided for sticking to the fastened band 112 through the design of Velcro® or a clasp (not shown in the figure).

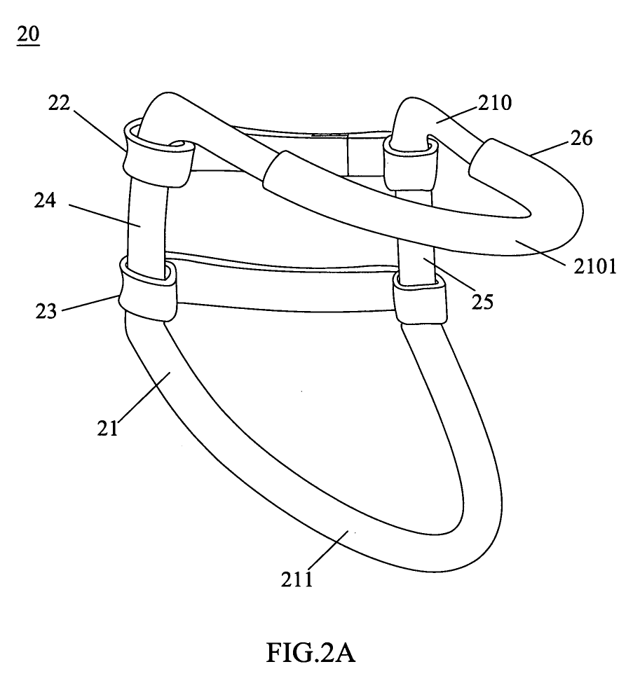

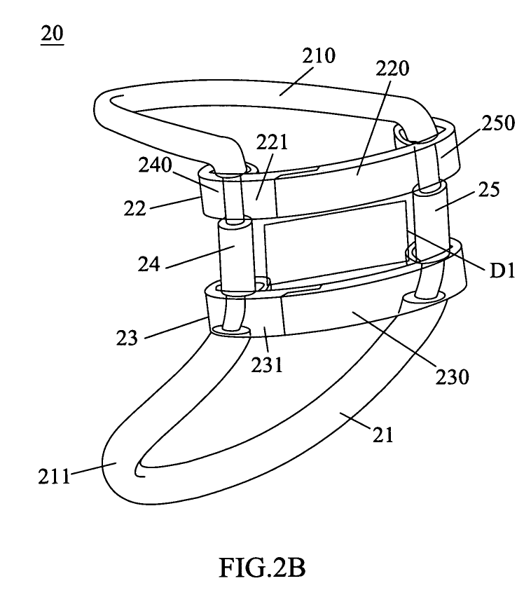

Referring to FIGS. 2A, 2B for the schematic diagrams illustrate a cervical collar 20 according to an embodiment of the preset invention. As shown in FIG. 2A, it is a side view illustrating the cervical collar 20, and the cervical collar 20 is composed of a main collar structure 21 and two fastening bands 22, 23. The main collar structure 21 is utilized to form a first loop 210 and a second loop 211. The first loop 210 is used to immobilize the patient's head illustrated in FIG. 1A. The front portion 2101 of the first loop 210 can be equipped with a soft mat 26 to make the chin of the patient more comfortable. The second loop 211 is configured to attach the upper chest and the shoulder of the patient illustrated in FIG. 1A in order to provide a support as the base. The main collar structure 21 further has two longitudinal portions 24, 25 between the first and second loop 210, 211. The longitudinal portions 24, 25 are attached at the back of the neck of the patient as illustrated in FIG. 1B. The longitudinal portions 24, 25 can be entwined by the fastening bands 22, 23 at the back of the neck through the concave portions 240, 250, so as to secure the main collar structure 21. It should be noted that the fastening bands 22, 23 is Velcro®, and can be repeated used, and can be adjusted to fix the main collar structure 21 according to the neck circumference of every patient. The two fastening bands 22, 23 further have securing bands 220, 230 and secured bands 221, 231 respectively. Both the securing bands 220, 230 and the secured bands 221, 231 are circularly disposed to the concave portions 240, 250, and the securing bands 220, 230 are provided for sticking to the secured bands 221, 231 through the design of Velcro® or a clasp (not shown in the figure). As shown in FIG. 2B, by using the fastening bands 22, 23, the treatment region D1 (referred to dashed lines) illustrated in FIG. 1B is therefore obtained to help post-surgical treatment without any difficulty.

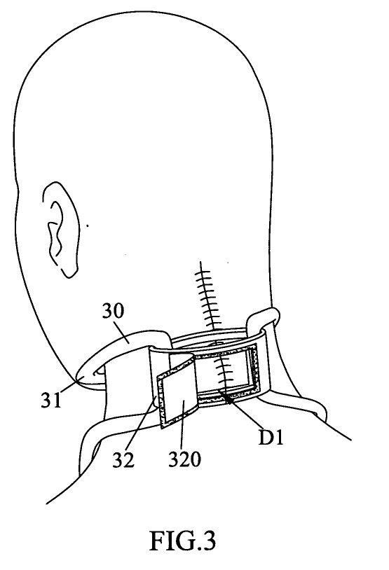

Moreover, the fastening bands can be designed to form a U-shaped as shown in FIG. 3. The main collar structure 31 of the cervical collar 30 can be equipped with the U-shaped fastening band 32. The doctors/nurses only need to tear off an adhesive portion 320 from the U-shaped fastening band 32 along the periphery of the U-shaped fastening band 32. The treatment region D1 illustrated in FIG. 1B could appear and the doctors/nurses can easily care wounds of the patient from the treatment region D1 after the surgical treatment is done. The U-shaped fastening band 32 is preferably Velcro®. It should be noted that the central area of the adhesive portion 320 does not have adhesion to avoid irritating the skins or wounds of the patient.

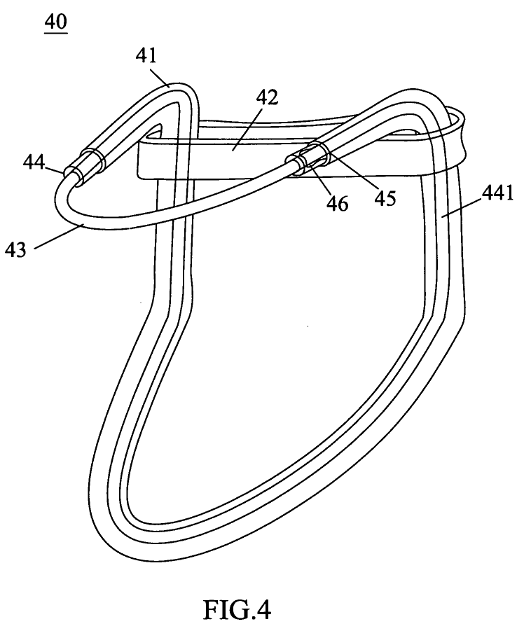

Referring to FIG. 4 for the structural diagram illustrates the cervical collar according to another embodiment of the present invention. The cervical collar 40 is applied to a patient's neck and encircles the neck between the patient's head and body to immobilize the patient's head and neck. The cervical collar 40 includes a main collar structure 41 and at least one fastening band 42. The main collar structure 41 is composed of a steel wire 43 made of cast iron, a rubber tube 44 having a hollow space 441 (as shown in dashed lines) and a nonwoven 45. The hollow space 441 is provided for accommodating the steel wire 43. The nonwoven 45 is adhered to a surface of the rubber tube 44 through an adhesive material 46 to comfortably attach the patient. The fastening band 42 is provided for securing the main collar structure at the back of the neck of the patient to form a treatment region D1 (as referred to dashed lines) at the back of the neck of the patient illustrated in FIG. 1B so that post-surgical treatment can be easily performed at the treatment region D1.

It should be noted that the materials and features of manufacturing the steel wire 43, the rubber tube 44, the nonwoven 45 and the fastening band 43 has been illustrated in FIG. 1A, 1B. The adhesive material 46 can be preferably resin or other materials with adhesion.

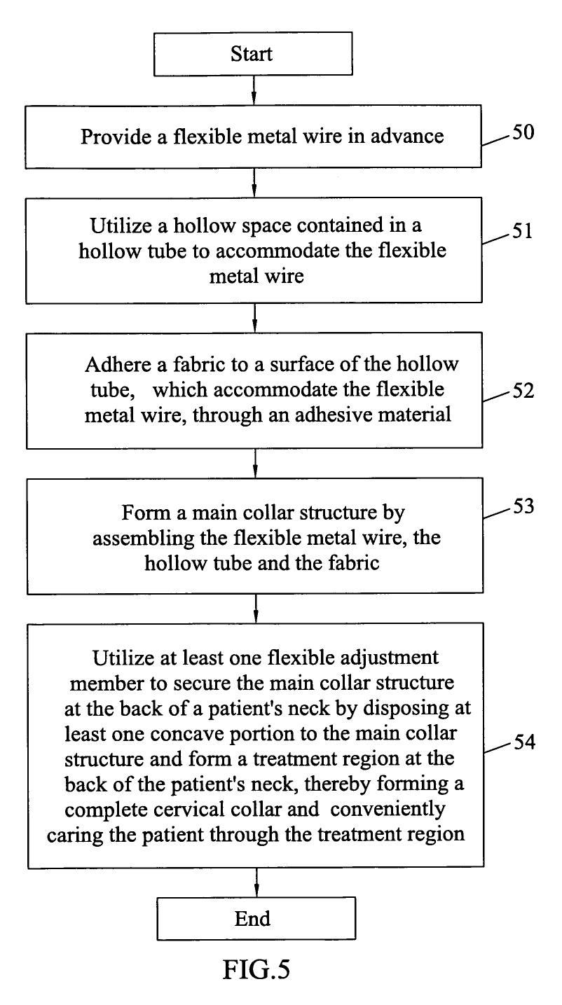

Referring to FIG. 5 for the flowchart illustrates a method of assembling a cervical collar according to an embodiment of the present invention. The method includes the following steps:

Step 50: Provide a flexible metal wire in advance;

Step 51: Utilize a hollow space contained in a hollow tube to accommodate the flexible metal wire;

Step 52: Adhere a fabric to a surface of the hollow tube, which accommodate the flexible metal wire, through an adhesive material;

Step 53: Form a main collar structure by assembling the flexible metal wire, the hollow tube and the fabric;

Step 54: Utilize at least one flexible adjustment member to secure the main collar structure at the back of a patient's neck by disposing at least one concave portion to the main collar structure and form a treatment region at the back of the patient's neck, thereby forming a complete cervical collar and conveniently caring the patient through the treatment region.

The flexible metal wire is preferably steel wire. The flexible metal wire can also be other kinds of metals with better flexibility. The steel wire can be preferably made of cast iron. The diameter of the flexible metal wire can be preferably between 0.5 mm (millimeter) and 7 mm. The hollow tube further has a hollow space that accommodates the flexible metal wire, and is made of rubber or other flexible material. The internal diameter of the hollow tube can be preferably between 4 mm and 10 mm, and the external diameter of the hollow tube can be preferably between 8 mm and 15 mm. The fabric can be preferably nonwoven or other material having waterproof function. The flexible adjustment member is preferably a fastening band, and can be Velcro® or other snap fasteners. The flexible adjustment member can be made of nylon, polymer composition material or other material capable of repeatedly adhering to and tearing off from the main collar structure.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A cervical collar for a patient's neck and helping post-surgical treatment, comprising:
a main collar structure configured to attach the upper chest and the shoulder of the patient and support the head of the patient, and the main collar structure having at least one concave portion; and
at least one flexible adjustment member disposed to the concave portions, and for securing the main collar structure at the back of the neck of the patient and configured to form a treatment region at the back of the neck of the patient, thereby conveniently caring the patient at the treatment region.

2. The cervical collar as claimed in claim 1, wherein the main collar structure is further composed of a flexible metal wire, a hollow tube and a fabric.

3. The cervical collar as claimed in claim 2, wherein the flexible metal wire is steel wire or other kinds of metals with better flexibility, and the steel wire is made of cast iron, and the diameter of the flexible metal wire is between 0.5 mm and 7 mm.

4. The cervical collar as claimed in claim 2, wherein the hollow tube further has a hollow space for accommodating the flexible metal wire, and the hollow tube is made of rubber or other flexible material.

5. The cervical collar as claimed in claim 2, wherein an internal diameter of the hollow tube is between 4 mm and 10 mm, and an external diameter of the hollow tube is between 8 mm and 15 mm.

6. The cervical collar as claimed in claim 2, wherein the fabric is nonwoven or other material having waterproof function.

7. The cervical collar as claimed in claim 1, wherein the flexible adjustment member can be made of nylon, polymer composition material or other material capable of repeatedly adhering to and tearing off from the main collar structure.

8. The cervical collar as claimed in claim 1, wherein the flexible adjustment member can be a fastening band, Velcro® or other snap fasteners.

9. A cervical collar for a patient's neck and encircling the neck between the patient's head and body to immobilize the patient's head and neck, comprising:
a flexible metal wire;
a hollow tube having a hollow space for accommodating the flexible metal wire; and
a fabric adhered to a surface of the hollow tube through an adhesive material to comfortably attach the patient;
wherein the flexible metal wire, the hollow tube and the fabric are assembled to form a main collar structure, and at least one flexible adjustment member is provided for securing the main collar structure at the back of the neck of the patient to form a treatment region at the back of the neck of the patient.

10. The cervical collar as claimed in claim 9, wherein the flexible metal wire is steel wire or other kinds of metals with better flexibility, and the steel wire is made of cast iron, and the diameter of the flexible metal wire is between 0.5 mm and 7 mm.

11. The cervical collar as claimed in claim 9, wherein the hollow tube further has a hollow space for accommodating the flexible metal wire, and the hollow tube is made of rubber or other flexible material.

12. The cervical collar as claimed in claim 9, wherein an internal diameter of the hollow tube is between 4 mm and 10 mm, and an external diameter of the hollow tube is between 8 mm and 15 mm.

13. The cervical collar as claimed in claim 9, wherein the fabric is nonwoven or other material having waterproof function.

14. The cervical collar as claimed in claim 9, wherein the flexible adjustment member can be made of nylon, polymer composition material or other material capable of repeatedly adhering to and tearing off from the main collar structure.

15. The cervical collar as claimed in claim 9, wherein the flexible adjustment member can be a fastening band, Velcro® or other snap fasteners.
